(19) United States
(12) Patent Application Publication
(10) Pub. No.: US 2017/0215986 A1
(43) Pub. Date: Aug. 3, 2017

(54) SURGICAL ACCESSORY AND METHODS OF USING THE SAME

(71) Applicants: Fredrick Brody, Bethesda, MD (US); Steve Eubanks, Winter Park, FL (US)

(72) Inventors: Fredrick Brody, Bethesda, MD (US); Steve Eubanks, Winter Park, FL (US)

(21) Appl. No.: 15/418,022

(22) Filed: Jan. 27, 2017

Related U.S. Application Data
(60) Provisional application No. 62/288,143, filed on Jan. 28, 2016.

(51) Int. Cl. A61B 90/70; B08B 9/045

(52) U.S. Cl. CPC A61B 90/70; B08B 9/045; A61B 2090/701

(57) ABSTRACT

A minimally invasive device that concurrently cleans trocars or cannulas and the lens of the scope and a method of using the same is disclosed. The disclosed device may include a rod having at least one end and at least one sponge attached to at least one end of the rod. There is also disclosed a method of cleaning trocars or cannulas and the lens of a minimally invasive scope concurrently with the device. The method may include the steps of attaching a first sponge to the rod and inserting one end of the device through a trocar such that at least one sponge is in contact with the inner surface of the trocar. The device may be removed from the trocar after sufficient cleaning is performed. If the device is inserted through a different trocar, the sponge end may be used to concurrently clean a scope lens while the scope is within a body cavity.

This application claims priority to U.S. Provisional Application No. 62/288,143, filed on Jan. 28, 2016, which is incorporated herein by reference in its entirety.

### TECHNICAL FIELD

The present disclosure relates generally to devices used to clean both the trocar and the lens of a scope used in minimally invasive surgery. The present disclosure also relates to methods of cleaning both the trocar and the lens of a scope used in minimally invasive surgery by inserting a rod through the trocar and the foam end conforms to the inner cannula of the trocar.

### BACKGROUND

The number of minimally invasive, robotic, and endoscopic procedures continue to increase since these types of procedures allow patients to heal faster with a shorter recovery time and a decreased risk for wound infections and hernias. These procedures entail small incisions to introduce instruments and scopes into various body cavities to perform a wide range of medical procedures that include ligating, cutting, suturing, and repairing tissue. However, these techniques require a continuous and clear image during the entire procedure. Typically, cannulas, trocars, or ports are placed through the small incisions and serve as portals to various body cavities. Due to the breadth of procedures, trocars are made of varying materials that range in length and diameter from one manufacturer to another.

Once the scope is inserted through a trocar, the scope lens may become obstructed due to smudging, condensation, or direct contact with pieces of tissue, bodily fluids, and condensation. These fluids include blood, bile, and fecal material. Also, pieces of tissue and fluid may be trapped within the trocars and collect on the lens as the scope is inserted into the body. Unfortunately, the field of view may be compromised numerous times during a procedure and prolong or hinder the progress of the procedure.

In order to provide the surgeon with a clear view, the scope needs to be removed from the body cavity, cleaned, and reinserted. During some procedures, it is not uncommon to remove and clean the scope 10-20 times which substantially increases the duration of the procedure and potentially leads to other complications. Thus, there is a need for a device that cleans the lens of the scope while the scope remains in a body cavity. As well, the device should concurrently clean the entire length of the trocar to ensure that tissue and fluid do not collect on the scope lens as it is inserted into the body.

The disclosed device and methods are directed to one or more of the problems set forth above and/or other problems of the prior art.

### SUMMARY

In one aspect, the present disclosure is directed to a minimally invasive device comprising a rod that may concurrently clean trocars or cannulas and the lens of the scope. The ability of the device to clean the trocar and lens is unique to this device.

In another aspect, the present disclosure is directed to a method of cleaning trocars or cannulas concurrently with the lens of a scope by using a minimally invasive device comprising a rod with two foam or sponge ends. In this embodiment, the method comprises attaching sponges to the rod; inserting one end of the device through the trocar such that at least one sponge is in contact with the inner surface of the trocar; and removing the device through the trocar after sufficient cleaning is performed. The same sponge or the other sponge (attached to the contralateral end of the rod) may be used to clean the lens of the scope while the scope is still within the body cavity.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a device according to the present disclosure.

FIG. 2 is another perspective of the device shown in FIG. 1.

FIG. 3 is a partially-sectioned, diagrammatic side view of an embodiment of a device constructed in accordance with principles of the present disclosure.

FIG. 4 is another perspective of the device shown in FIG. 3.

FIGS. 5(a) and 5(b) are partially-sectioned, diagrammatic side views of one end of a device constructed in accordance with principles of the present disclosure having a sponge that is screwed thereon.

FIG. 6 is a schematic of the sponge that can be used in an embodiment of a device constructed in according to the present disclosure.

FIG. 7 is a schematic showing threaded end of a rod according to the present disclosure.

FIG. 8 is a schematic of a device according to the present disclosure.

FIG. 9 is a schematic of a device according to the present disclosure.

FIG. 10 is a schematic of a device according to the present disclosure.

### DETAILED DESCRIPTION

As used herein, “minimally invasive” refers to a surgical procedure that is performed through tiny incisions instead of one large opening. During a minimally invasive procedure, surgeons make several small incisions in the skin—just a few millimeters, in some cases. There are described devices used in such procedures that can pass through one of the incisions. These devices are call trocars or cannulas. The trocars act as a portal to the body cavity as they traverse into the body cavity.

There is disclosed devices used to clean both the trocar and the lens of a scope used in minimally invasive surgery. In an embodiment, the device comprises a rod with foam or sponges that may be attached to both ends. The two-headed device can clean a variety of trocar sizes and various scopes. In an embodiment, the minimally invasive device described herein concurrently cleans trocars or cannulas and the lens of the scope.

There is also disclosed a method of cleaning trocars or cannulas of a scope concurrently with the lens of a scope by using the minimally invasive device described herein. In an embodiment, the method comprises cleaning both the trocar and the lens of a scope used in minimally invasive surgery by inserting a rod through the trocar and the foam end conforms to the inner cannula of the trocar. The rod is gently slid through the trocar and debris or fluid is pushed out of the trocar or absorbed by the sponge. Concurrently, the foam end may be used to clean the lens of the scope while the scope is still in the body cavity. Different shapes of foam with varying lengths and diameters are available for both ends of the rod. This variety maximizes the utility of the two-headed rod.

In one embodiment, the method may comprise attaching a first sponge to the rod, inserting one end of the device through the trocar such that at least one sponge is in contact with the inner surface of the trocar. Once the device is engaged within the trocar, the rod is grasped and used to clean the inner cannula. The device is removed after sufficient cleaning is performed. The scope may then be reinserted through the trocar. If debris or fluid is still within the trocar, the cleaning procedure is repeated. If the lens becomes dirty, the rod with the attached sponge may also be used to enter another trocar and clean the lens of the minimally invasive scope without removing the scope from the body cavity. The detachable sponges may be exchanged multiple times during the procedure if the sponge becomes dirty or damaged. The sponges are detachable and new sponges may be exchanged multiple times during the procedure.

A device is described to clean a wide range of trocars regardless of diameter or length. The device includes a rod with two variegated ends. Sponges or a variety of foam shaped structures may be attached and detached to the ends of the rod. These sponges are available in various lengths, widths, and shapes to accommodate a wide range of minimally invasive and robotic trocars and procedures. Once the device is inserted through the trocar, the sponges can be used to clean the scope lens.

Numerous types of sponges may be attached and detached to the rod as needed to ensure that the trocar and lens remain clean during the entire surgical procedure. The scope and trocar cleaning device may consist of a rod with a sponge or foam connected to each end. The figures and text describe the rod and sponge in more detail. The varying degree of sizes and types of sponges allow the device to clean a wide range of trocars while exchanging the foam ends during the procedure if the sponge becomes dirty. Numerous manufacturing companies make a variety of different trocars with varying diameters. Different size sponges may be attached to the rod to accommodate the various trocars. Regardless of size, the same sponge may be used to clean the scope lens.

The rod may be made of a hard plastic, silicone, stainless steel, or various alloys. The rod may be used as a disposable or a reusable device depending on the type of material. In one embodiment, the length of the rod should extend 10 cm, at a minimum. As well, rods may extend over 50 cm for longer trocars.

The rod may have a circular circumference but may also have a hexagonal circumference to enhance tactile feedback and twirling of the device to clean the trocar and lens. Alternatively, various external circumferences or shapes of the rod may be used to augment twirling of the rod as it is inserted and manipulated.

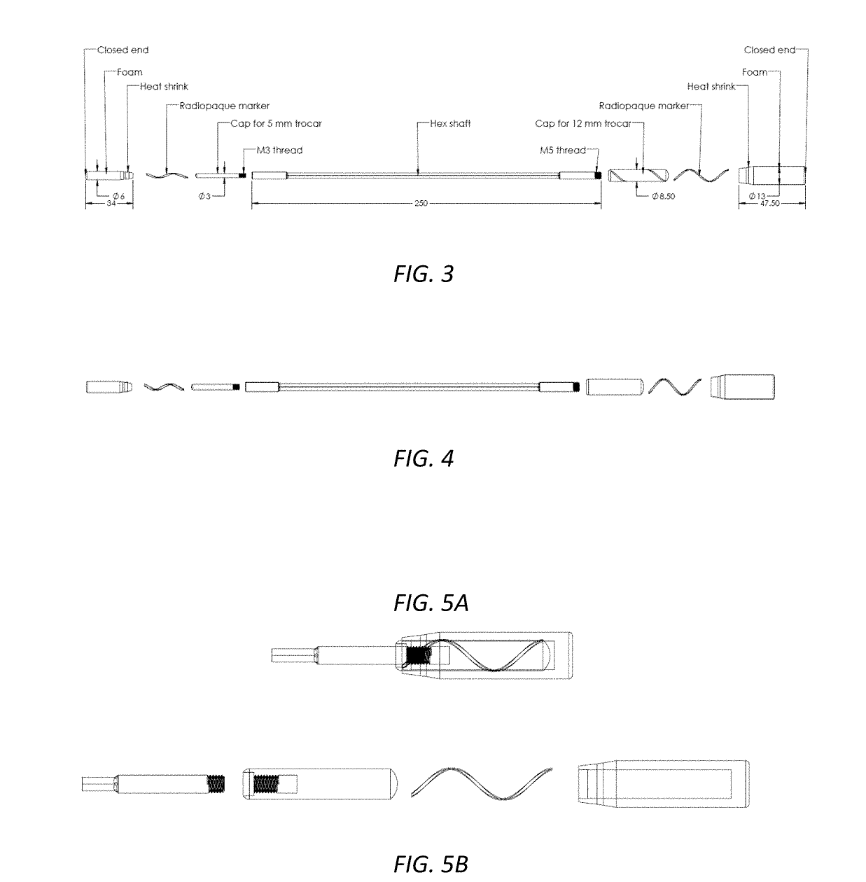

The ends of the rod may include threads, clips, hooks, or variegated edges that facilitate a wide range of sponge attachments. As shown in FIGS. 3, 4 and 5, the ends of the rod may be threaded so sponges may be screwed onto the end of the rod. FIG. 3 particularly demonstrates two different ends of the rod. The ends of the rod may be receptive or threaded. Clips or hooks may be used at the end of the rod and sponges may be hooked or clipped onto the end of the rod. Also, various snaps and matching edges may be used to attach the sponge to the end of the rod. This type of configuration allows new sponges to be used during a single operative procedure.

With further reference to FIG. 3, an X-ray detectable strip may be attached, embedded and/or wrapped around the rod if the rod is made of a material that is not X-ray detectable. In one embodiment, the X-ray detectable strip may consist of a piece of wire or thread that extends the entire length of the rod or along specific segments of the rod. With further reference to this embodiment in FIG. 3, the radiopaque strip that may be positioned between sponge and the trocar to allow the surgeon to visualize the positioning and orientation of lens cleaner when positioned within the body cavity. The outer wall of the trocar may include a groove configured to receive a strip. The groove may have a width and depth substantially equal to or greater than the width and depth of the strip to allow the strip to be recessed into the outer wall of trocar. This position of the strip allows the pad to maintain an unobstructed absorbent surface and prevents any scratching from contact between the strip and the lens of the scope. As depicted in FIG. 3, the strip and groove may each have a helical configuration extending circumferentially around the rod. The helical configuration of the strip allows visualization of the lens cleaner at any angle. However, it is also contemplated that the strip may have other configurations such as linear or serpentine (not shown).

In other embodiments, the strip may be embedded within the sponge. In other embodiments, the strip may be formed from radiopaque markings or dyes applied to the sponge. Additional strips may be provided to allow the surgeon to enhance visualization of lens cleaner through X-ray, fluoroscopy, MRI, and/or CT scan imaging.

In various embodiments, the sponge may be made of foam, cotton, or an absorptive material that can gently clean the lens of a scope. The material should be sturdy enough to clean the inside of the trocar. The sponge may be manufactured into various shapes such as a cylinder, sphere, hexagon, or ellipse to enhance trocar cleaning.

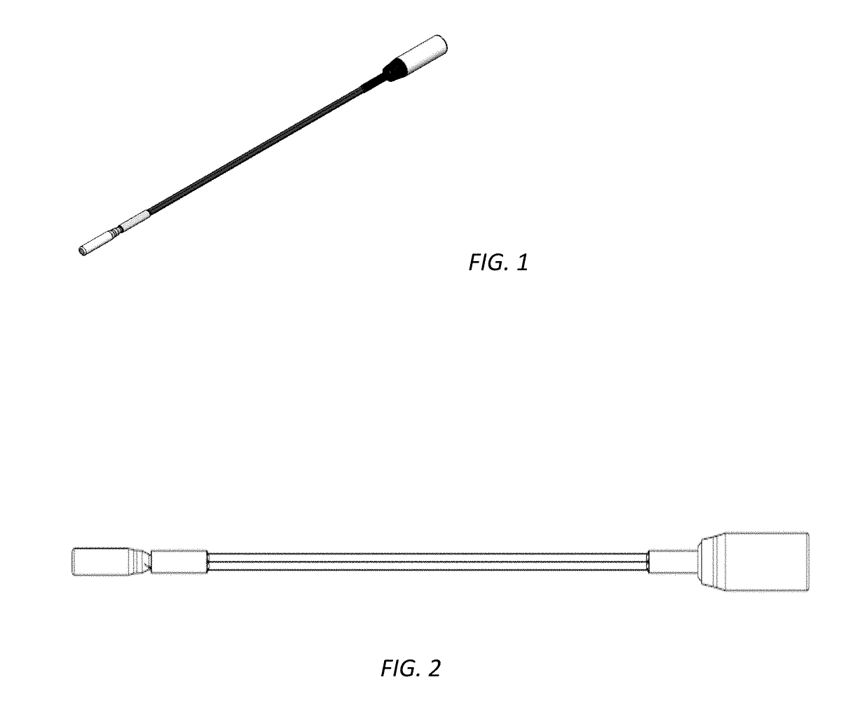

The length and diameter of the sponge includes a range of sizes to accommodate various trocar diameters and lengths. In one embodiment, the sponge is at least 1 cm in length and may extend to 4 cm in length. The diameter of the sponge may range from 2 mm to 50 mm, such as from 5 mm to 40 mm, 10 mm to 30 mm, or 15 to 25 mm. Since the rod entails two ends, two different or two similar sizes of sponge may be attached to each respective end of the rod. FIGS. 1 and 2 show rods with different size sponges and FIGS. 9 and 10 show rods with similar size sponges.

The sponge material can compress as it is inserted through a cannula or trocar. The sponge material would then expand slightly as it exits the trocar and extends into the body cavity. The trocar tip and undersurface of the trocar tip are cleansed as the sponge expands when it exits the trocar and enters the body cavity.

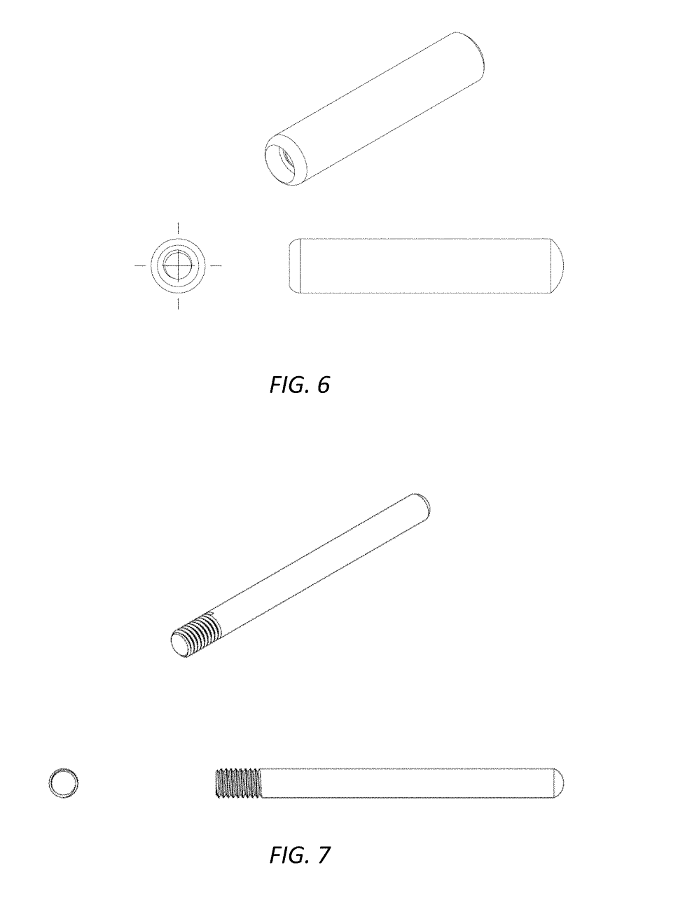

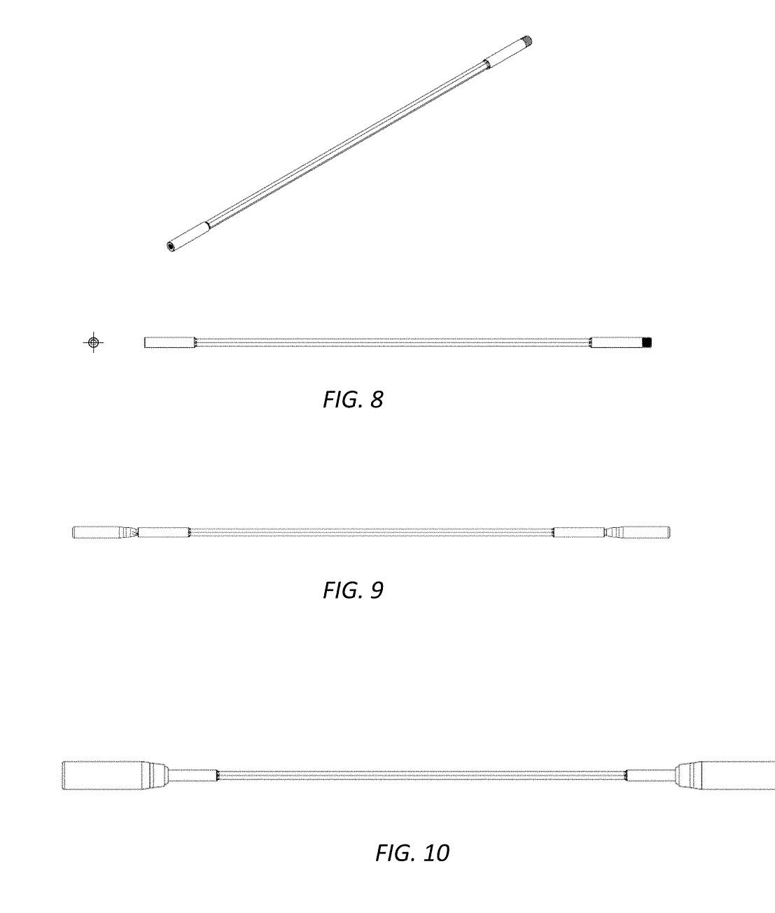

The sponges may have corresponding adaptive couplers, threads, or cored inlets to attach seamlessly to the ends of the rod. The rod may extend into the center of the sponge or entirely through the sponge to ensure adequate support and structural integrity. As shown in FIGS. 5, 6, and 7, the sponge can include a cap that inserts directly into the sponge and then onto the rod as well. As shown in FIGS. 4, 5, 6, and 7, the cap may have a receptive end for a threaded rod or the cap may be threaded to attach to the receptive end of the rod.

An X-ray detectable strip is embedded or wrapped around the sponge. This strip may help locate the sponge if it becomes detached in the body. The strip may be a piece thread, wire, or sheet of X-ray detectable material.

An exemplary method may be disclosed herein. During an exemplary minimally invasive procedure, the lens of the scope inevitably becomes smudged or dirty and obscures the surgeon's view. The scope does not need to be removed from its respective trocar. The two-headed rod is used through a second trocar. The diameter and length of the second trocar is grossly measured and a corresponding sponge is attached to the rod. Two slightly different sponges or similar sponges may be used depending on the trocar or type of debris or fluid on the lens. The unique ability of this device allows the scope lens to be cleaned without removing the scope from the body cavity.

During an exemplary minimally invasive procedure, the device is inserted through a second trocar while the scope remains in the body. The sponge on one end of the rod is inserted through the trocar and directed towards the lens of the scope. Simultaneously, the scope can visualize the device as it enters the body cavity through the tip of the trocar. The scope is held in place but is directed towards the sponge. The sponge is then used to wipe the lens gently to remove fluid, tissue, or condensation from the lens. This action may be repeated multiple times to obtain a clear field of vision.

Alternatively, the rod and corresponding sponge may be held steady and the scope may be advanced slowly towards the sponge. The scope may then be wiped gently against a stationary sponge to clear any debris from the field of vision. The entire device may be removed from the second trocar and flipped 180 degrees along its long axis and the sponge at the opposite end of the rod may be used in a similar fashion to clean the lens of the scope. As well, the entire device may be removed from the trocar and the sponge may be removed and replaced with a clean sponge or a sponge with a different shape or contour. The capacity to remove and add new sponges enables the device to continual clean a dirty or smudged lens.

If debris or fluid accumulates within the trocar, the device may be used to clean the inner tunnel of the trocar. The sponge at the tip of the rod is inserted through the center of the trocar and slowly introduced through the entire length of the trocar. The rod may be twirled or twisted as the sponge is inserted to ensure that the entire tract of the trocar is cleaned. The sponge is then removed from the trocar and the scope may be introduced through the clean inner tunnel of the trocar. If the trocar still contains tissue or fluid, the sponge may be reinserted. If the sponge is dirty, then the opposite end of the device may be inserted. Alternatively, the sponge may be detached and a new clean sponge may be attached to the end of the rod.

As depicted herein, the unique capacity of this device embodies both a trocar cleaning device and a device to simultaneously clean a scope lens during a minimally invasive or robotic procedure. The method of cleaning may be performed during any surgery involving a scope. For example, the method may be applicable to procedures performed laparoscopically, thoracoscopically, endoscopically, and robotically.

Other types of cleaning devices that might be in combination with the disclosed device are described in U.S. Published Appl. No. 2016/0022367, which is herein incorporated by reference. The device described in this comprises a sleeve including an inner surface configured to engage a medical device; and a pad secured around the sleeve and configured to wipe the lens of the scope.

It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for the features of interest, but not to exclude such from the scope of the disclosure entirely unless otherwise specifically indicated.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

What is claimed:

1. A unique minimally invasive device that concurrently cleans trocars or cannulas and the lens of the scope, comprising:
a rod having at least one end; and
at least one sponge attached to the at least one end of the rod.

2. The minimally invasive device of claim 1, wherein the rod is at least 10 cm in length.

3. The minimally invasive device of claim 1, wherein the rod may be as long as 100 cm in length.

4. The minimally invasive device of claim 1, wherein the circumference of the rod includes a shape to enhance tactile feedback.

5. The minimally invasive device of claim 4, wherein the shape to enhance tactile feedback is chosen from the group consisting of circular, hexagonal, and square.

6. The minimally invasive device of claim 1, wherein the rod is made from a material chosen from the group consisting of plastic, silicone, stainless steel, and metal alloys.

7. The minimally invasive device of claim 1, wherein the rod is disposable or reusable.

8. The minimally invasive device of claim 6, wherein the rod includes an X-ray detectable strip, thread, or wire that is embedded along or within the rod.

9. The minimally invasive device of claim 1, wherein the rod further comprises exactly two ends and at least one sponge is attached to each end of the rod.

10. The minimally invasive device of claim 1, wherein at least one end of the rod is threaded and at least one sponge is configured to screw onto at least one end of the rod.

11. The minimally invasive device of claim 1, wherein at least one end of the rod comprises a clip or hook and at least one sponge is configured to couple to at least one end of the rod.

12. The minimally invasive device of claim 1, wherein a major component of the sponge is chosen from the group consisting of foam, microfiber material, cotton, or a soft synthetic material that will not scratch the minimally invasive or robotic lens of a scope.

13. The minimally invasive device of claim 1, wherein the sponge has a diameter ranging from about 2 mm to 3 cm.

14. The minimally invasive device of claim 1, wherein the at least one sponge has a length ranging from about 1 cm to 4 cm.

15. The minimally invasive device of claim 1, wherein at least one sponge has a shape chosen from the group consisting of cylinders, hexagons, and squares.

16. The minimally invasive device of claim 1, wherein at least one sponge comprises a material that is configured to be selectively compressible to allow at least one sponge to fit firmly into and through various trocars.

17. The minimally invasive device of claim 1, wherein at least one end of the rod is threaded and at least one sponge includes a cored center with threads that are configured to screw onto at least one end of the rod.

18. The minimally invasive device of claim 1, wherein at least one sponge includes at least one clip or variegated end that is configured to clasp or hook to the rod.

19. The minimally invasive device of claim 1, wherein the rod includes two ends, at least one sponge is attached to each end, and the sponges are of the same size or are different sizes.

20. The minimally invasive device of claim 1, wherein the sponge includes an X-ray detectable wire, thread, or strip embedded along an axis of the sponge or attached to an outer surface of the sponge.

21. A method of cleaning trocars or cannulas concurrently with a lens of a scope by using a minimally invasive device including a rod, the method comprising:
attaching at least one sponge to the rod;
inserting one end of the device through a trocar such that at least one sponge is in contact with an inner surface of the trocar;
removing the device through the trocar after sufficient cleaning is performed; and
using the sponge to concurrently clean the lens while the scope is within the body cavity.

22. The method of claim 21, wherein upon introduction into the trocar, the device undergoes at least one of the following motions plunging, twisting or twirling to facilitate cleaning of the inner surface of the trocar.

23. The method of claim 21, further including:
attaching a second sponge to the rod at an opposite end of the rod relative to a first sponge.

24. The method of claim 21, further including rotating the device about 180 degrees along a major axis of the rod to allow the second sponge to clean the inner surface of the trocar.

25. The method of claim 21, further including introducing the device through a second trocar.

26. The method of claim 25, wherein the scope is directed towards the second trocar but held in place.

27. The method of claim 25, wherein the device is directed towards the lens of the scope under direct vision.

28. The method of claim 25, further including:
applying pressure to the sponge when it contacts the lens to allow it to wipe away debris, tissue, condensation, or combinations thereof, from the lens.

29. The method of claim 25, further including:
attaching a second sponge to the rod at an opposite end of the rod relative to the first sponge; and
rotating the device 180 degrees along a major axis of the rod to allow the second sponge to clean the inner surface of the trocar.

30. The method of claim 21, further including exchanging the sponge if the sponge becomes dirty or damaged.
